(19) United States
(12) Patent Application Publication
(10) Pub. No.: US 2016/0000413 A1
(43) Pub. Date: Jan. 7, 2016

(54) OPTICALLY AUGMENTED FINE NEEDLE ASPIRATION BIOPSY DEVICE AND METHOD OF USING THE SAME

(71) Applicants: TRUSTEES OF BOSTON UNIVERSITY, Boston, MA (US); BOSTON MEDICAL CENTER CORPORATION, Boston, MA (US); FRAUNHOFER USA, INC., Plymouth, MI (US)

(72) Inventors: John C. BRIGGS, Lexington, MA (US); Andre SHARON, Newton, MA (US); Alexis SAUER-BUDGE, Lincoln, MA (US); David CHARGIN, Newton, MA (US); Irving BIGIO, Chestnut Hill, MA (US); Jennifer ROSEN, Wellesley, MA (US); Ousama A'AMAR, Dedham, MA (US); Aaron SIZE, Waltham, MA (US); Stephanie LEE, Newton, MA (US)

(21) Appl. No.: 14/763,778

(22) PCT Filed: Jan. 30, 2014
(86) PCT No.: PCT/US14/13804

§ 371 Date: Jul. 27, 2015

Related U.S. Application Data
(60) Provisional application No. 61/759,748, filed on Feb. 1, 2013.

(51) Int. Cl. A61B 10/02; A61B 5/00

(52) U.S. Cl. CPC A61B 10/0283; A61B 5/0084

(57) ABSTRACT

Devices for performing fine-needle aspiration of tissue and methods of using the same are disclosed. The device includes a syringe including a fiber optic probe, a plunger, and a needle and a holder including a coupling mechanism, a first actuator, and a second actuator. The fiber optic probe is configured to move within a lumen of the needle and is guided into the needle by a fiber optic channel. The plunger is configured to create a vacuum in the needle.

### CROSS REFERENCE TO RELATED APPLICATIONS

This application claims benefit under 35 U.S.C. §119(e) of the U.S. Provisional Application No. 61/759,748, filed Feb. 1, 2013, the content of which is incorporated herein by reference in its entirety.

### FIELD OF THE INVENTION

The present invention relates to fine needle aspiration biopsies and, more particularly, to identifying and aspirating desired tissue to be biopsied.

### BACKGROUND OF THE INVENTION

Fine needle aspiration (“FNA”) biopsy (“FNAB”) is the standard evaluation of a thyroid nodule of a patient. Ultrasound guidance has been used to image the target tissue and guide the needle of a syringe to the tissue to be biopsied. Once the needle is guided to the tissue to be biopsied, a plunger of the syringe is moved to create a vacuum that aspirates the tissue. Diagnostic determination of the tissue requires a certain number of cells. If insufficient cells are aspirated, the biopsy procedure must be performed again, usually 2-6 months later.

It is estimated that more than 600,000 FNAB procedures are performed each year in the United States. The number of biopsies that yield insufficient cells to make a diagnostic determination ranges from 7 to 17%. Even in an elite academic thyroid center using ultrasound guidance, insufficient FNA biopsies occurred in 17% of 3,589 nodules biopsied.

Even if the needle is disposed in a position to aspirate the desired cells at the beginning of aspiration, manual manipulation of the device is required for aspiration and typically results in axial movement of the needle. This axial movement can result in insufficient biopsies and/or cells being aspirated from non-desired tissue.

The American Thyroid Association, as well as most endocrinologists and endocrine surgeons, recommend a total or near-total thyroidectomy for well-differentiated thyroid cancer. A total thyroidectomy allows for better use of Iodine-131 (¹³¹I) for postsurgical treatment because of a statistically significant improved overall survival and decreased recurrence rate. Because patients with “indeterminate” thyroid nodules are not known to have the certain diagnosis of cancer preoperatively, clinical decision making incorporates surgical judgment. Most surgeons prefer to treat patients with indeterminate nodules as though they did in fact have a cancer. A total or near-total thyroidectomy is performed as the initial operation in these situations, which subjects these patients to all of the risks of thyroidectomy. These risks include permanent bilateral recurrent laryngeal nerve injury which requires a permanent tracheotomy and permanent hyperparathyroidism which requires calcium supplementation, having clinical consequences, as well as the need for life-long thyroid-hormone replacement treatments. The present invention is directed to solving these and other problems.

### SUMMARY OF THE INVENTION

In accordance with one embodiment, a device for performing fine-needle aspiration of tissue includes a syringe and a holder. The syringe includes a fiber optic probe, a plunger, and a needle. The fiber optic probe is configured to move within a lumen of the needle. The fiber optic probe is guided into the needle by a fiber optic channel. The plunger is configured to create a vacuum in the needle. The holder includes a coupling mechanism, a first actuator, and a second actuator. The coupling mechanism coupling the syringe in a fixed position relative to the holder. The first actuator being configured to extend the fiber optic probe into the needle and to retract the fiber optic probe from the needle. The second actuator is configured to extend the plunger thereby creating a vacuum within the needle.

In accordance with another embodiment, a method for performing fine-needle aspiration of tissue includes the steps of inserting a needle having a fiber optic probe disposed therein into tissue of a patient including tissue to be biopsied, generating an optical spectrum of tissue disposed proximate to a distal end of the needle, determining a spectral signature of the proximate tissue using the optical spectrum, removing the fiber optic probe from the needle, and aspirating the proximate tissue by moving a plunger to create vacuum in the needle.

In accordance with yet another embodiment, a device for performing fine-needle aspiration of tissue includes a syringe and a holder. The syringe includes a fiber optic channel, a barrel, and a needle. The fiber optic channel has a fiber optic probe disposed for movement therein. The barrel has a plunger disposed for movement therein. The needle has a lumen extending from a distal end to a proximal end of the needle. The proximal end of the lumen is operatively connected to both the fiber optic channel and the barrel. The lumen is configured to receive the fiber optic probe. The holder is configured to receive the syringe and includes a first actuator and a second actuator. The first actuator configured to extend the fiber optic probe from the fiber optic channel and into the lumen, and further configured to retract the fiber optic probe from the lumen. The second actuator is configured to slide the plunger, thereby causing material to move from the distal end of the lumen toward the barrel.

In accordance with still yet another embodiment, a device for performing fine-needle aspiration of tissue includes a biopsy needle, a fiber optic probe, and an actuator. The biopsy needle has a hollow core and is mounted to a handle. The biopsy needle includes a distal end adapted for insertion into tissue and a proximal end adapted for coupling to the handle. The biopsy needle is coupled to a vacuum source whereby vacuum can be selectively applied to the biopsy needle. The fiber optic is probe coupled to the biopsy needle and adapted to translate through the hollow core of the biopsy needle to the distal end and to be retracted from the biopsy needle. The actuator is coupled to the fiber optic probe to move the fiber optic probe through the hollow core of the biopsy needle and retract the fiber optic probe from the hollow core of the biopsy needle.

### BRIEF DESCRIPTION OF THE DRAWINGS

The invention may best be understood by reference to the following description taken in conjunction with the accompanying drawings, in which:

FIG. 1 illustrates a side view of a fine needle aspiration biopsy device with a fiber optic probe in a deployed position and a plunger in a first position according to an embodiment;

FIG. 2 illustrates a cross-sectional view of the fine needle aspiration biopsy device of FIG. 1 with the fiber optic probe in the deployed position and the plunger in the first position according to an embodiment;

FIG. 3 illustrates a side view of the fine needle aspiration biopsy device of FIG. 1 with the fiber optic probe in a retracted position and the plunger in a second position according to an embodiment;

FIG. 4 illustrates a flow diagram of a method for aspirating desired tissue according to an embodiment;

FIG. 5A illustrates a fine needle aspiration biopsy syringe with a fiber optic probe according to an embodiment;

FIG. 5B illustrates a holder configured to receive configured to receive the syringe of FIG. 4A;

FIG. 6A illustrates the holder of FIG. 5B in an open position, showing the interior;

FIG. 6B illustrates a perspective view of the syringe of FIG. 5A from a second view;

FIG. 7A illustrates an end-view of a fiber optic probe according to an embodiment;

FIG. 7B illustrates an end-view of a fiber optic probe according to another embodiment.

### DETAILED DESCRIPTION OF ILLUSTRATED EMBODIMENTS

Although the invention will be described in connection with certain preferred embodiments, it will be understood that the invention is not limited to those particular embodiments. On the contrary, the invention is intended to cover all alternatives, modifications, and equivalent arrangements as may be included within the spirit and scope of the invention as defined by the appended claims.

It has been determined that one common reason for cellular insufficiency during an FNA thyroid biopsy is cystic degeneration of the nodule. Cystic degeneration results in a fibrous nodule with small pockets that have degenerated into fluid. This fluid volume may be in pockets that are sub-millimeter in size. At this size, the resolution of the ultrasound machine is too coarse to distinguish the difference between the tissue and the fluid pockets. Thus, even when the FNAB device is guided by real-time ultrasound imaging, the FNAB device may be sampling fluid rather than tissue.

Optical tissue diagnosis mediated by fiber-optic probes can be used to perform noninvasive or minimally invasive real time assessment of tissue pathology in situ. One method of performing this assessment can include the use of elastic scattering spectroscopy (“ESS”). ESS provides a point spectroscopic measurement of subcellular micromorphology and cellular composition of a tissue, using electromagnetic radiation in a broad wavelength range (e.g., 320-900 nm in one example system). Markers include size and hyperchromaticity of cell nuclei, chromatin granularity, nuclear crowding, and changes in the size and/or density of mitochondria and other cellular organelles. ESS spectra derive from the wavelength-dependent optical scattering efficiency and the effects of changes in the scattering phase function (e.g., the angular probability distribution for scattering), caused by the optical index gradients (e.g., due to cellular and subcellular structures). Normal and abnormal tissues generate different scattering spectral signatures as a result of changes in nuclear size, density, granularity of the chromatin, mitochondrial swelling, and other subcellular structural changes. These different scattering spectral signatures represent the optical-spectroscopy equivalent of histological appearances. The ESS method senses those morphology differences in a semi-quantitative manner, without actually imaging the microscopic structure. An important advantage of ESS is that it may provide a real-time objective and semi-quantitative assessment of tissue pathology that may not require on-site special expertise and/or subjective image interpretation as in conventional histopathology. ESS is also convenient: the unit used is lightweight and mobile, and the ESS probe can fit through a conventional 21-23 gauge needle for a minimally invasive diagnosis. A measurement is rendered in less than 250 ms and can be triggered by, for example, a simple foot pedal. Examples of ESS systems capable of performing this assessment are described in papers and publications listed in the ESS References section below, which are hereby incorporated by reference in their entirety.

Advantageously, an FNAB device incorporating an ESS probe disposed near the tip of the needle is able to report whether the needle tip is sensing fluid or solid tissue. For example, the probe can be guided to a nodule using ultrasound imaging, record optical spectra, and be repositioned within the nodule until the returned optical spectrum indicates solid tissue disposed proximate to the needle tip.

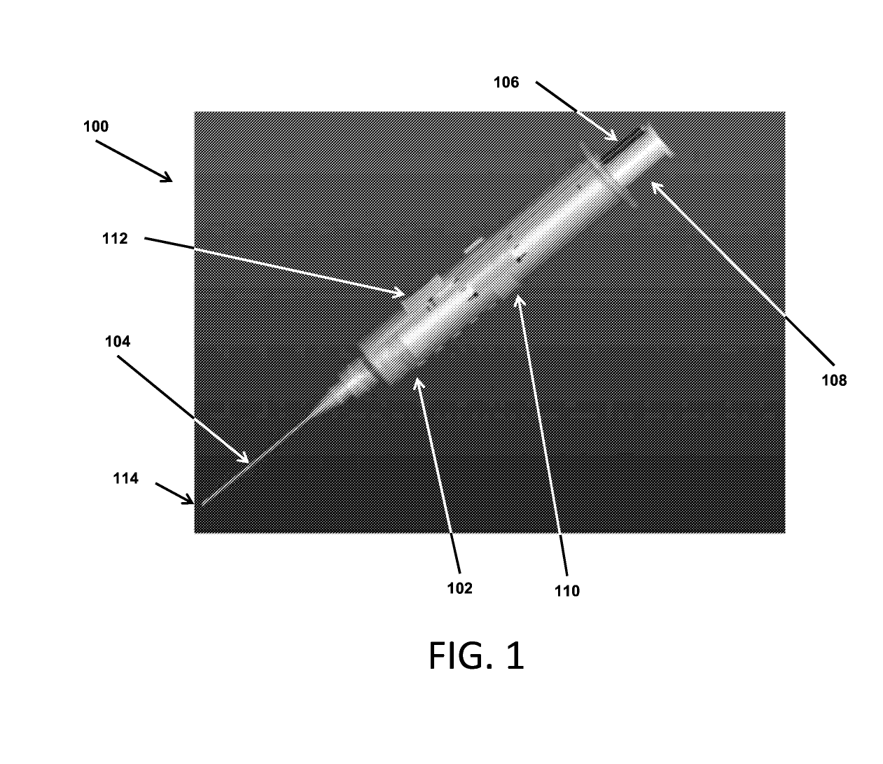

FIG. 1 illustrates a side view of a fine needle aspiration biopsy (FNAB) device 100 including a barrel 102, a needle 104, a fiber optic probe 106, a plunger 108, a locking mechanism 110, and a slide mechanism 112. The needle 104 is coupled to the barrel 102 and is configured to pierce tissue. The needle 104 includes a tip 114 distal to the barrel 102 and a lumen extending from the tip 114 to the barrel 102, allowing for passage of material along the length of the needle 104.

The fiber optic probe 106 can move within the lumen of the needle 104. The slide mechanism 112 is coupled to the fiber optic probe 106 and is configured to slide the distal end of the fiber optic probe between a deployed position and a retracted position. Fine needle aspiration (FNA) needles typically range from 23 gauge to 27 gauge. More preferably, the needle 104 is 25 gauge. The fiber optic probe 106 is flexible and includes fiber optic cable. In order for the fiber optic probe 106 to move within the lumen of the needle, the outer diameter of the fiber optic probe 106 must be less than the inner diameter of the needle, but should not be so small that binding occurs when the fiber optic probe 106 is pushed from the proximate end. For example, a fiber optic probe 106 with an outer diameter of 346 μm can be used with a 23 gauge needle having an inner diameter of 370 μm.

As used herein, “distal end” refers generally to a portion of the object nearer to a patient while in use and “proximal end” refers generally to a portion of the object nearer to a practitioner, unless otherwise specified. For example, a distal end of a needle is the end that pierces a patient's skin and a proximal end of the needle is the end that connects to the barrel of a syringe.

When in the deployed position, the distal end of the fiber optic probe 106 is disposed near the tip 114. When in the retracted position, the fiber optic probe 106 is disposed such that material near the tip 114 may be aspirated through the needle 104. In the illustrated embodiment, the fiber optic probe 106 is disposed in the deployed position.

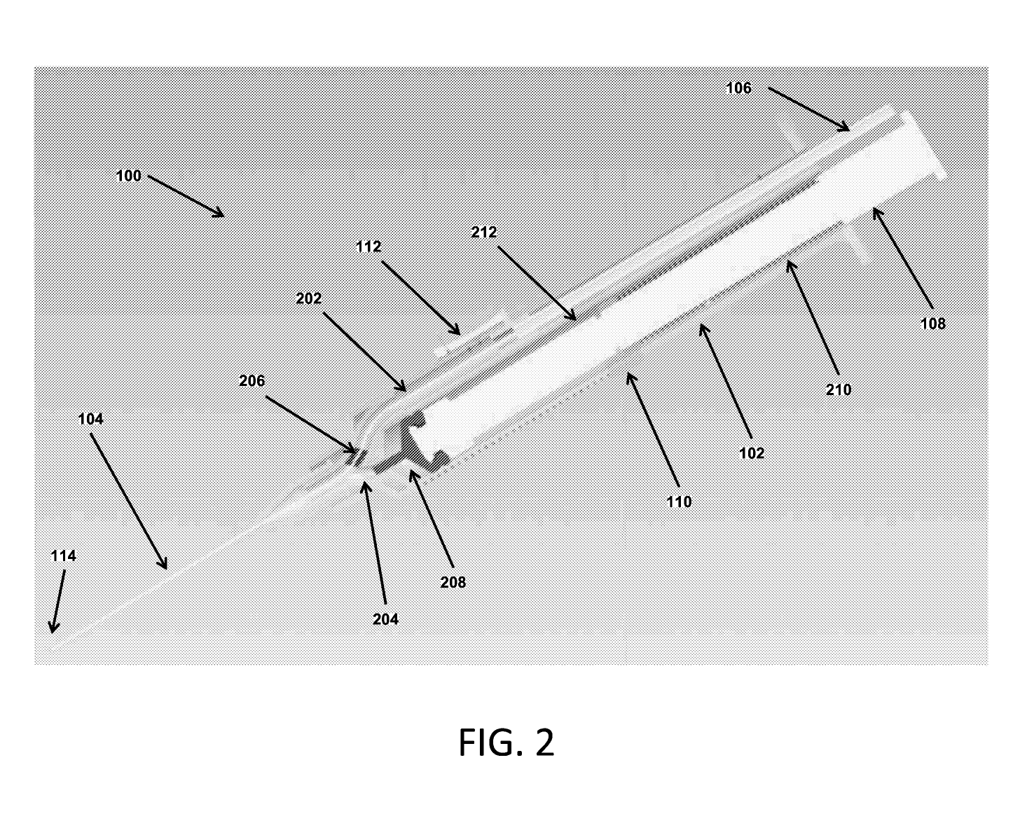

The plunger 108 slides within a plunger chamber (FIG. 2) and forms a substantially airtight seal with an inner wall. The locking mechanism 110 selectively engages the plunger 108 to prevent movement of the plunger 108 in at least one direction.

FIG. 2 illustrates a cross-sectional view of the FNAB device 100. As shown, the needle 104 lumen is coupled to both a fiber optic channel 202 and an aspiration channel 204. The fiber optic channel 202 guides the fiber optic probe 106 into the needle 104 as the fiber optic probe 106 is slid toward the deployed position. Sealing mechanism 206 forms a substantially airtight seal between the fiber optic probe 106 and the fiber optic channel 202 so that aspiration is not hindered by the incorporation of the fiber optic probe 106.

The aspiration channel 204 is between the needle 104 and a plunger chamber 208. Movement of the plunger 108 creates negative or positive pressure in the plunger chamber 208 because of the airtight seal between the plunger 108 and plunger chamber 208. The aspiration channel 204 communicates these pressures to the needle 104. Thus, outward movement of the plunger 108 creates negative pressure in the plunger chamber 208, which creates negative pressure at the tip 114. The negative pressure at the tip 114 results in a vacuum that aspirates material disposed proximate to the tip 114. The plunger 108 includes a spring 210 that biases the plunger 108 outward. The plunger 108 also includes a locking channel 212 that is selectively engaged by locking mechanism 110. When engaged, locking channel 212 prevents both inward and outward movement of the plunger 108.

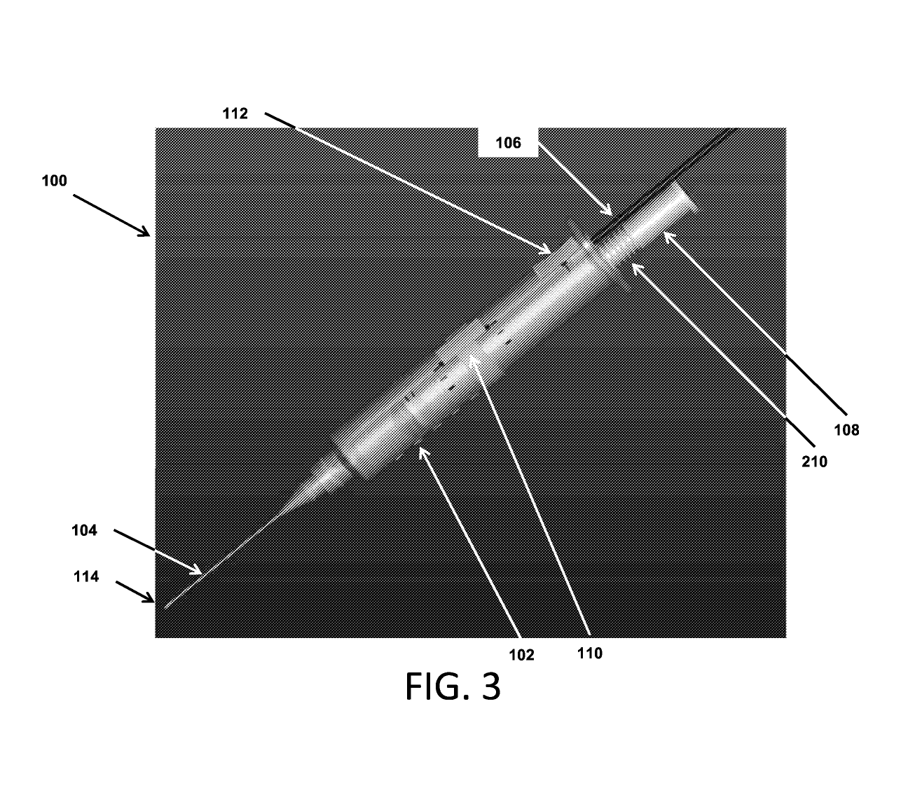

FIG. 3 illustrates a side view of the FNAB device 100 with the fiber optic probe 106 in the retracted position and the plunger 108 in the second position. The slide mechanism 112 is disposed at a proximate end of the barrel. When the slide mechanism 112 is disposed in this position, the fiber optic probe 106 is in the retracted position and does not interfere with aspiration of material through the needle 104. The locking mechanism 110 is disposed in a disengaged position where the locking channel 212 is not engaged by the locking mechanism 110. The plunger 108 may move inwardly or outwardly when the locking mechanism 110 is in the disengaged position. As shown, the plunger is disposed at the second position because the spring 210 outwardly biases the plunger 108.

The FNAB device 100 is configured to be held and operated substantially with a single hand. The device 100 can be gripped with a “pencil grip” where the barrel 102 is grasped generally between the practitioner's thumb, index finger, and middle finger. Advantageously, the pencil grip and single-handed operation minimizes accidental movement of the tip 114. In the illustrated embodiment, the device 100 is designed to be grasped in the practitioner's right hand. The locking mechanism 110 is disposed in a location where it can be easily actuated from the engaged position to the disengaged position by the thumb of the practitioner. Additionally, accidental axial movement of the tip 114 occurs when the practitioner has to manually move the plunger 108. This is typically accomplished by the practitioner grasping the barrel 102 with one hand and the plunger 108 with the other, then moving the hands in opposite directions. The forces exerted by each hand fluctuate, leading to accidental axial movement of the tip 114. Use of the spring 210 to move the plunger 108 decreases the accidental axial movement of the tip 114 during aspiration because the force exerted is internal to the device 100 and evenly placed.

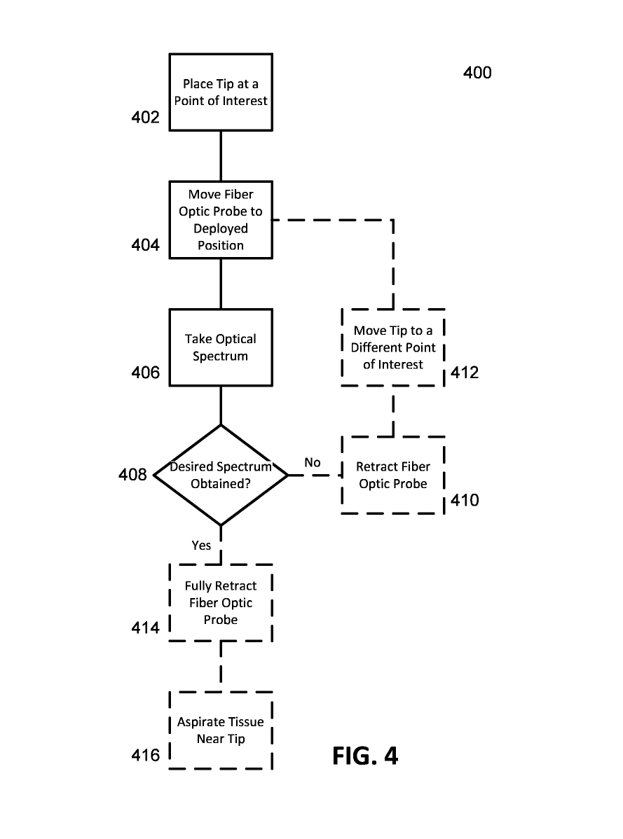

FIG. 4 illustrates a flow diagram of a method 400 for aspirating desired tissue. The method 400 begins at step 402 where the tip 114 is placed at a point of interest such as a thyroid nodule. Placement of the tip 114 can be assisted by real-time ultrasound guidance. When the tip 114 is disposed at the point of interest, the fiber optic probe 106 is extended from within the lumen of the needle 104, proximate the tip 114, to the deployed position at step 404. An optical spectrum is then taken at step 406. The taking may be initiated by a number of different methods including in response to a foot pedal or button actuated by the practitioner, in response to the optical probe 106 reaching the deployed position, or in response to a predetermined amount of time elapsing. The obtained optical spectrum is compared to a desired spectrum or to a plurality of desired spectra at decision box 408. If the obtained optical spectrum does not match the desired spectrum, the fiber optic probe 106 is retracted into the lumen of the needle 104 at step 410. The tip 114 is moved to a new point of interest at step 412 and steps 404, 406, and 408 are repeated until a desired spectrum is obtained. Step 410 is optional as indicated by the broken-line box. Thus, the tip 114 can be moved to a new point of interest at step 412 and then steps 406 and 408 are repeated without retracting the fiber optic probe 106. It is contemplated that the fiber optic probe 106 may be shaped so as to not inhibit movement of the tip 114 though the flesh. In one nonlimiting example, this is accomplished by shaping the fiber optic probe 106 to be flush across an opening of the lumen when the fiber optic probe 106 is in the deployed position.

When a desired spectrum is obtained, the fiber optic probe 106 is fully retracted to the retracted position at step 414. The desired tissue is then aspirated at step 416. The plunger 108 is moved outwardly, creating negative pressure in the plunger chamber 208. The negative pressure aspirates tissue disposed proximate the tip 114 into the plunger chamber through the needle 104 and aspiration channel 204. After the sample is taken, the device is removed from the tissue and the sample of aspirated tissue is subjected to diagnostic tests.

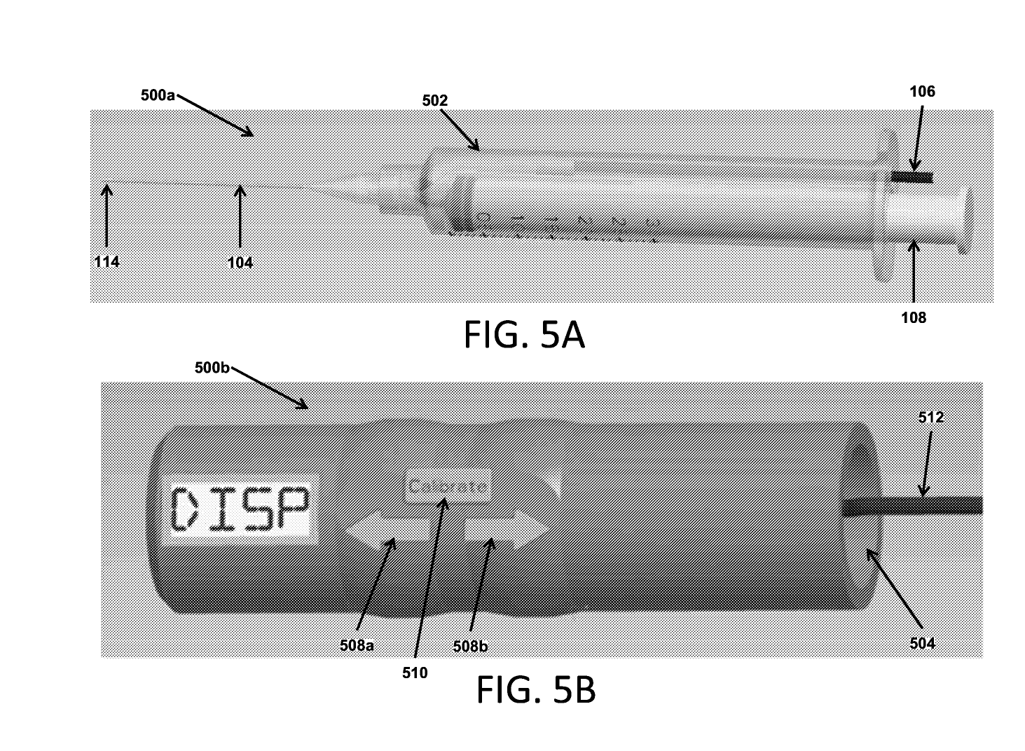

FIGS. 5A and 5B illustrate an FNAB device 500 including a syringe 500a and a holder 500b. The syringe 500a includes a barrel 502, a needle 104, a fiber optic probe 106, and a plunger 108. Similarly to the FNAB device 100 described above, the fiber optic probe 106 can be moved within the lumen of the needle 104 and can be guided into the needle using fiber optic channel 202. Also as described above, plunger 108 is configured to create a vacuum in the needle 104 in order to aspirate tissue disposed proximate to the needle 104. The holder 500b receives the syringe 500a and actuates components thereon. The holder includes a bore 504 therein that receives the syringe 500a. The outer surface of the bore includes a display 506a actuator-control buttons 508a, b and a calibration button 510. The display 506 is used to convey information to the practitioner such as calibration information, configuration information, placement of the fiber optic probe or plunger, error information, and/or information related to the tissue disposed near the tip. The display 506 can include a liquid crystal display, a plurality of indicators, or any other device suitable to convey desired information to a practitioner. The holder 500b can also include a data transmission cable 512 for transferring information to and from the device 500. In one nonlimiting example, the data transmission cable 512 can be coupled to a controller that analyzes received optical spectra and the display 506 indicates to the practitioner whether the tip 114 is disposed proximate to desired tissue to be biopsied.

Actuator-control buttons 508a, b are used to control an actuator or actuators that move the fiber optic probe 106 and/or the plunger 108. In the illustrated embodiment, the forward button 508a controls motion of both the fiber optic probe 106 and the plunger 108 toward the tip 114 and the rearward button 508b controls motion of both the fiber optic probe 106 and the plunger 108 away from the tip 114. In this embodiment, the plunger 108 and the fiber optic probe 106 are operatively coupled in order to prevent the fiber optic probe 106 and the plunger 108 from interfering with each other. Beneficially, the sequential process of the technique allows for automatic rejection of inadvertent actuations of the buttons 508a,b, 510 and provides both visual and auditory feedback to the practitioner for the mistaken button press. Alternatively, four buttons may be provided where the first and the second control the inward and outward movement of the fiber optic probe 106 and the third and the fourth control inward and outward movement of the plunger 108.

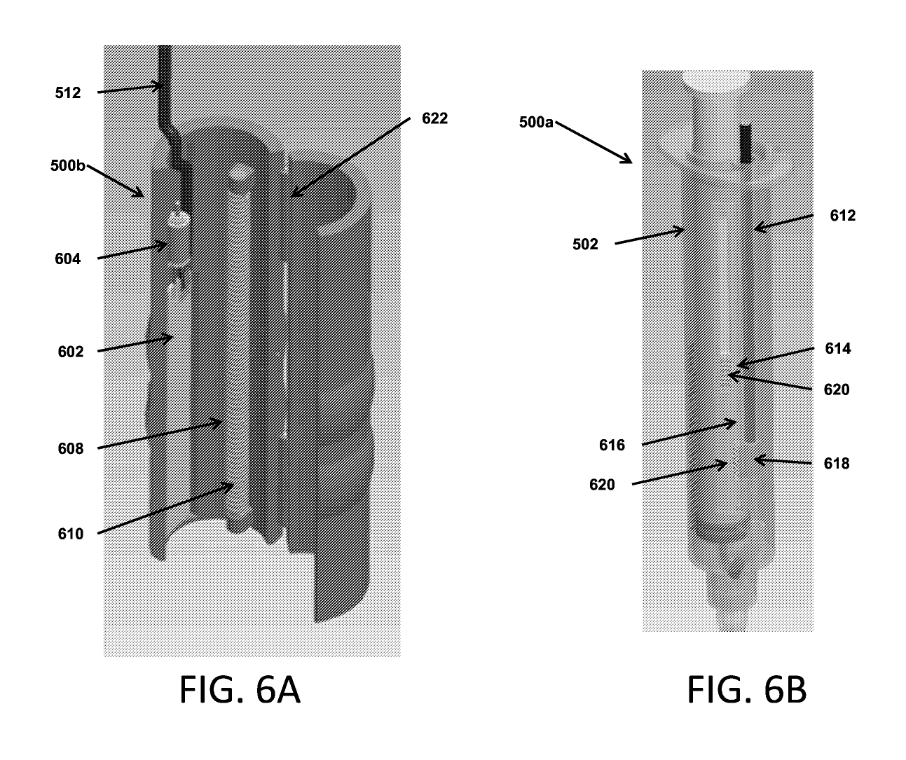

FIG. 6A illustrates the holder 500b in an open position. The holder 500b can include the data transmission cable 512, circuit board 602, motor 604 and lead screw 606. The circuit board 602 is operatively connected to the data transmission cable 512 and controls the relay of information between the device 500 and a remote device such as a controller. The circuit board 602 is also coupled to the display 506 and controls the information displayed thereon. The circuit board 602 is further coupled to the buttons 508a,b, 510 and the motor 604. When one of actuator-control buttons 508a,b is depressed, the circuit board 602 passes signals to the motor 604. The motor 604 is mechanically coupled to the lead screw 608 via a drive mechanism (not shown). The drive mechanism translates motion of the motor 604 to the lead screw 608. The lead screw 608 includes threading 610 thereon. It is contemplated that other translation mechanisms may be used in place of or in addition to the motor and lead screw such as a rack and pinion driven by a motor, a motor and worm gear, etc.

FIG. 6B illustrates a perspective view of the syringe 500a including a plunger guide-slot 612, plunger half-nut 614, probe guide-slot 616, and probe half-nut 618. The plunger half-nut 614 and the probe half-nut 618 each have threading 620 thereon that engage the threading 610 of the lead screw 608. The plunger half-nut 614 is disposed on the plunger 108 and extends through plunger guide-slot 612. The plunger guide-slot 612 guides movement of the plunger half-nut 614 and isolates the motion to the axial direction. The probe half-nut 618 is disposed on the fiber optic probe 106 and extends through the probe guide-slot 616. The probe guide-slot 616 guides movement of the probe half-nut 618 and isolated the motion to the axial direction.

The syringe 500b is coupled to the holder 500a by placing the syringe 500b within the bore 504 of the holder. The syringe 500b is placed such that the threading 620 of the plunger half-nut 614 and the probe half-nut 618 engage the threading 610 of the lead screw 608. The plunger half-nut 614 engages the lead screw 608 and moves laterally along a line parallel to the axis of the lead screw 608. The probe half-nut 618 engages the lead screw 608 and moves laterally along a line parallel to the axis of the lead screw 608 that is rotated about 90° from the line of the plunger half-nut 614. The line of the probe half-nut 618 is rotated from the line of the plunger half-nut 614 so that movement of the fiber optic probe 106 does not interfere with movement of the plunger 108. The holder 500b is closed by pivoting about hinges 622. When closed, a friction-fit is formed between the syringe 500a and the holder 500b that prevents movement of the barrel 502 relative to the bore 504. It is contemplated that other forms of actuation may be used. In some embodiments, the fiber optic probe 106 is driven by a first actuator and the plunger 108 is driven by a second actuator, separate from the first.

With reference to FIG. 4 above, the method of using the electronic FNAB device 500 is similar to using the mechanical FNAB device 100. The tip 114 is place at a point of interest at step 402. The fiber optic probe is moved to the deployed position at step 404 by depressing the forward button 508a. The optical spectrum is taken at step 406 and analyzed at step 408. If a desired spectrum is not obtained, the rearward button 508b is depressed and the fiber optic probe is retracted slightly before moving the tip to a new point of interest at step 412. If a desired spectrum is obtained, the fiber optic probe 106 is fully retracted at step 414 by continuing to depress and hold the rearward button 508b. The desired tissue is aspirated at step 416 by continuing to depress and hold the rearward button 508 such that the plunger 108 moves outward.

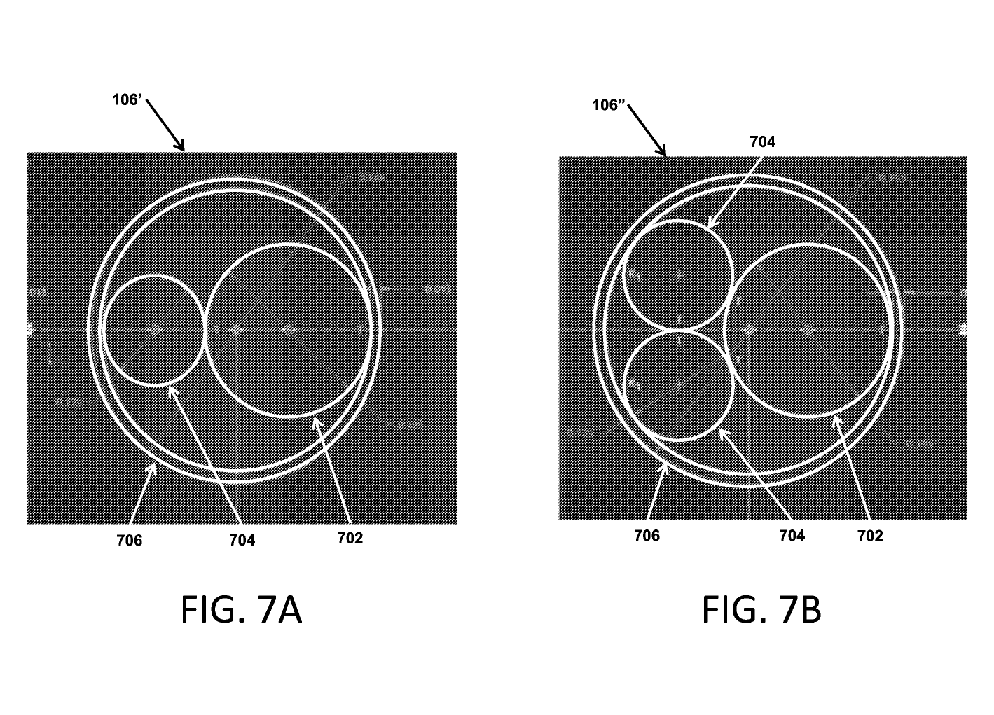

FIG. 7A illustrates an end-view of a fiber optic probe 106′ that can be used in accord with the present concepts. The fiber optic probe 106′ has fiber optic cables including an illumination cable 702 and a collection cable 704. The fiber optic probe 106′ is connected to a measurement system. The measurement system emits light through the illumination cable 702 and receives light to be analyzed through the collection cable 704. In one nonlimiting example, the illumination cable 702 measures about 195 μm in diameter and the collection cable 704 measures about 125 μm in diameter. The thickness of a wall 706 of the fiber optic probe 106′ measures about 13 μm. Thus, the fiber optic probe 106′ measures about 346 μm in diameter.

FIG. 7B illustrates an end-view of a fiber optic probe 106″ that can be used in accord with the present concepts. The fiber optic probe 106″ has fiber optic cables including an illumination cable 702 and two collection cables 704. In one nonlimiting example, the illumination cable 702 measures about 195 μm in diameter and the collection cables 704 each measure about 125 μm in diameter. The thickness of a wall 706 of the fiber optic probe 106′ measures about 13 μm. The most closely packed arrangement of the illumination cable 702 and the two collection cables 704 includes the illumination cable 702 contacting both collection cables 704 and each collection cable 704 contacting the other collection cable 704 and the illumination cable 702. In this configuration, the overall diameter of the fiber optic probe 106″ is about 355 μm. Advantageously, the fiber optic probe 106″ with two collection cables 704 can collect twice as much light as the fiber optic probe 106′ with only one collection cable 704 while only minimally increasing the overall diameter.

In one example device, the fiber optic probe included fiber optic cables with approximately 125 μm center-to-center separation. The illumination cable was approximately 150 μm in diameter and the collection cable was approximately 100 μm in diameter. The probe is sensitive to both scattering and absorption properties in the tissue because of the small separation of the fiber optic cables. An example UV-visible spectroscopic system used a pulsed broadband Xenon arc lamp as the light source. The light source resulted in a useful system response that covered a spectral range of about 320 nm to about 800 nm. Five measurements were taken on each specific site in the thyroid gland during ultrasound-guided FNAB. The five measurements were then averaged to represent the ESS spectrum for that site. The procedure was repeated in 3 to 6 sites in the gland; however, the cells were aspired only from the last site, which was used to correlate ESS measurements with the cytological assessment. Measurements were taken in the presence of ambient room illumination with background subtraction. The spectral response of each system was calibrated by recording a reference spectrum from a spectrally-flat diffuse reflector SPECTRALON® (Labsphere, Inc., North Sutton, N.H.). Thus, the displayed spectra were calculated according to:

$$I ( λ ) = \frac{{I ( λ )}_{tissue} - {I ( λ )}_{tissue background}}{{I ( λ )}_{reference} - {I ( λ )}_{reference background}}$$

In some embodiments, optical techniques other than ESS are employed to distinguish whether desired tissue to be aspirated is disposed near the tip. Nonlimiting examples of these techniques include use of optical spectroscopy such as fluorescence spectroscopy, Raman spectroscopy, and other forms of diffuse reflectance spectroscopy.

Surprisingly, integration of an ESS probe with FNA device yielded a very low rate of insufficient biopsies. In one example, 92 biopsies resulted in only 3 insufficient biopsies. This equates to an insufficiency rate of 3.3%, which is lower than any insufficiency rate in any published series.

Advantageously, systems, methods, and devices in accord with the disclosed concepts can reduce the number of insufficient biopsies. An insufficient biopsy is estimated to occur in more than 100,000 individuals each year. The reduced insufficiency rate would result in an estimated savings of $50 million annually.

In some embodiments, devices in accord with the present concepts are completely disposable. In some embodiments, the holder is reusable and the syringe is disposable, including the fiber optic probe. Beneficially, this combination lowers cost per use because the circuitry and components of the holder are typically too costly to be disposable while the syringe can be produced relatively inexpensively. In this embodiment, the holder can be easily disinfected between procedures without substantial disassembly. Additionally, the disposable nature of the syringe eliminates the need for on-site sterilization leading to safer procedures and increased clinical acceptability.

In some embodiments, the device does not include a plunger. In these embodiments, negative pressure and aspiration are created by a vacuum pump. The vacuum can be actuated by, for example, a foot pedal provided to the practitioner or a button incorporated on the device.

While the concepts disclosed herein have been described with reference to thyroid nodules, it is contemplated that devices and techniques can be beneficial in analyzing liver cancer, probing pulmonary lymph nodes, identifying the parathyroid during laparoscopic surgery, analyzing other solid organ pathologies.

It will be evident to those skilled in the art that the invention is not limited to the details of the foregoing illustrated embodiment and that the present invention may be embodied in other specific forms without departing from the spirit or essential attributes thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein.

### ESS REFERENCES

1. Mourant J R, Bigio I J, Boyer J D, Johnson T M, Lacey J, Bohorfoush Iii A G, Mellow M H. Elastic scattering spectroscopy as a diagnostic tool for differentiating pathologies in the gastrointestinal tract: preliminary testing. J Biomed Opt. 1996; 1(2):192-9. PubMed PMID: 23014685.
2. Mourant J R, Johnson T M, Los G, Bigio I J. Non-invasive measurement of chemotherapy drug concentrations in tissue: preliminary demonstrations of in vivo measurements. Phys Med Biol. 1999; 44(5):1397-417. PubMed PMID: 10368027.
3. Johnson K S, Chicken D W, Pickard D C, Lee A C, Briggs G, Falzon M, Bigio I J, Keshtgar M R, Bown S G. Elastic scattering spectroscopy for intraoperative determination of sentinel lymph node status in the breast. J Biomed Opt. 2004; 9(6):1122-8. PubMed PMID: 15568931.
4. A'Amar O M, Ley R D, Bigio I J. Comparison between ultraviolet-visible and near-infrared elastic scattering spectroscopy of chemically induced melanomas in an animal model. J Biomed Opt. 2004; 9(6):1320-6. PubMed PMID: 15568954.
5. Sharwani A, Jerjes W, Salih V, Swinson B, Bigio I J, El-Maaytah M, Hopper C. Assessment of oral premalignancy using elastic scattering spectroscopy. Oral oncology. 2006; 42(4):343-9. PubMed PMID: 16321565.
6. Dhar A, Johnson K S, Novelli M R, Bown S G, Bigio I J, Lovat L B, Bloom S L. Elastic scattering spectroscopy for the diagnosis of colonic lesions: initial results of a novel optical biopsy technique. Gastrointestinal endoscopy. 2006; 63(2):257-61. PubMed PMID: 16427931.
7. Lovat L B, Johnson K, Mackenzie G D, Clark B R, Novelli M R, Davies S, O'Donovan M, Selvasekar C, Thorpe S M, Pickard D, Fitzgerald R, Fearn T, Bigio I, Bown S G. Elastic scattering spectroscopy accurately detects high grade dysplasia and cancer in Barrett's oesophagus. Gut. 2006; 55(8):1078-83. PubMed PMID: 16469795; PubMed Central PMCID: PMC1856278.
8. Zhu Y, Fearn T, Mackenzie G, Clark B, Dunn J M, Bigio I J, Bown S G, Lovat L B. Elastic scattering spectroscopy for detection of cancer risk in Barrett's esophagus: experimental and clinical validation of error removal by orthogonal subtraction for increasing accuracy. J Biomed Opt. 2009; 14(4):044022. PubMed PMID: 19725733; PubMed Central PMCID: PMC2849300.
9. Mulvey C S, Sherwood C A, Bigio I J. Wavelength-dependent backscattering measurements for quantitative real-time monitoring of apoptosis in living cells. J Biomed Opt. 2009; 14(6):064013. PubMed PMID: 20059251; PubMed Central PMCID: PMC2794414.
10. Keshtgar M R, Chicken D W, Austwick M R, Somasundaram S K, Mosse C A, Zhu Y, Bigio I J, Bown S G. Optical scanning for rapid intraoperative diagnosis of sentinel node metastases in breast cancer. The British journal of surgery. 2010; 97(8):1232-9. PubMed PMID: 20593429.
11. Austwick M R, Clark B, Mosse C A, Johnson K, Chicken D W, Somasundaram S K, Calabro K W, Zhu Y, Falzon M, Kocjan G, Fearn T, Bown S G, Bigio I J, Keshtgar M R. Scanning elastic scattering spectroscopy detects metastatic breast cancer in sentinel lymph nodes. J Biomed Opt. 2010; 15(4):047001. PubMed PMID: 20799832; PubMed Central PMCID: PMC2917446.
12. Suh H, A'Amar O, Rodriguez-Diaz E, Lee S, Bigio I, Rosen J E. Elastic light-scattering spectroscopy for discrimination of benign from malignant disease in thyroid nodules. Ann Surg Oncol. 2011; 18(5):1300-5. PubMed PMID: 21174158.
13. Calabro K, Curtis A, Galarneau J R, Krucker T, Bigio I J. Gender variations in the optical properties of skin in murine animal models. J Biomed Opt. 2011; 16(1):011008. PubMed PMID: 21280895.
14. Mulvey C S, Zhang K, Liu W H, Waxman D J, Bigio I J. Wavelength-dependent backscattering measurements for quantitative monitoring of apoptosis, part 1: early and late spectral changes are indicative of the presence of apoptosis in cell cultures. J Biomed Opt. 2011; 16(11):117001. PubMed PMID: 22112133; PubMed Central PMCID: PMC3221717.
15. Mulvey C S, Zhang K, Bobby Liu W H, Waxman D J, Bigio I J. Wavelength-dependent backscattering measurements for quantitative monitoring of apoptosis, part 2: early spectral changes during apoptosis are linked to apoptotic volume decrease. J Biomed Opt. 2011; 16(11):117002. PubMed PMID: 22112134; PubMed Central PMCID: PMC3221718.
16. A'Amar O M, Liou L, Rodriguez-Diaz E, De Las Morenas A, Bigio I J. Comparison of elastic scattering spectroscopy with histology in ex vivo prostate glands: potential application for optically guided biopsy and directed treatment. Lasers in medical science. 2012. PubMed PMID: 23247663.
17. Mourant J R, Bigio I J, Boyer J D, Johnson T M, Lacey J, Bohorfoush A G, Mellow M H. Elastic scattering spectroscopy as a diagnostic tool for differentiating pathologies in the gastrointestinal tract: preliminary testing. Journal of Biomedical Optics. 1996; 1(2):192.
18. Lee A C, Pickard C D O, Briggs G M, Bigio I, Falzon M, Keshtgar M R S, Bown S G. Elastic scattering spectroscopy for intraoperative detection of sentinel lymph node metastasis. European Journal of Cancer. 2002; 38:S59-S. PubMed PMID: ISI:000175052800090.
19. Lee A C, Pickard C D O, Keshtgar M R S, Briggs G M, Falzon M, Lakhani S, Bigio I, Bown S G. Elastic scattering spectroscopy for the diagnosis of breast cancer. British Journal of Surgery. 2002; 89:74-. PubMed PMID: ISI:000176322800219.
20. Lovat L B, Johnson K, Mackenzie G D, Clark B R, Novelli M R, Davies S, O'Donovan M, Selvasekar C, Thorpe S M, Pickard D, Fitzgerald R, Fearn T, Bigio I, Bown S G. Elastic scattering spectroscopy accurately detects high grade dysplasia and cancer in Barrett's oesophagus. Gut. 2006; 55(8):1078-83. PubMed PMID: ISI:000239073000009.
21. Keshtgar M R S, Chicken D W, Austwick M R, Somasundaram S K, Mosse C A, Zhu Y, Bigio I J, Bown S G. Optical scanning for rapid intraoperative diagnosis of sentinel node metastases in breast cancer. British Journal of Surgery. 2010; 97(8):1232-9. PubMed PMID: ISI:000280361600010.
22. Boyer J, Mourant J, Bigio I. Theoretical and experimental demonstrations of elastic scattering spectroscopy as a diagnostic for tissue pathologies. Alfano R, editor. Washington D C: Optical Society of America; 1994.

1. A device for performing fine-needle aspiration of tissue, the device comprising:
a syringe including a fiber optic probe, a plunger, and a needle, the fiber optic probe being configured to move within a lumen of the needle, the fiber optic probe being guided into the needle by a fiber optic channel, the plunger configured to create a vacuum in the needle; and
a holder including a coupling mechanism, a first actuator, and a second actuator, the coupling mechanism coupling the syringe in a fixed position relative to the holder, the first actuator being configured to extend the fiber optic probe into the needle and to retract the fiber optic probe from the needle, the second actuator being configured to extend the plunger thereby creating a vacuum within the needle.

2.-41. (canceled)
